(19) United States
(12) Patent Application Publication
(10) Pub. No.: US 2017/0266336 A1
(43) Pub. Date: Sep. 21, 2017

(54) Colour-stable superabsorbent

(71) Applicant: BASF SE, Ludwigshafen (DE)

(72) Inventors: Matthew Gande, Charlotte, NC (US); Kristine Hartnagel, Schifferstadt (DE); Katarzyna Dobrosielska-Oura, Ludwigshafen (DE); Felicitas Leonie Sperling, Mannheim (DE)

(73) Assignee: BASF SE, Ludwigshafen (DE)

(21) Appl. No.: 15/455,739

(22) Filed: Mar. 10, 2017

(30) Foreign Application Priority Data
Mar. 15, 2016 (EP) 16160360.0

(51) Int. Cl. A61L 15/60; B01J 20/26; A61L 15/24; A61L 15/18; C07D 211/94; A61L 15/20

(52) U.S. Cl. CPC A61L 15/60; C07D 211/94; A61L 15/20; A61L 15/24; A61L 15/18; B01J 20/267

(57) ABSTRACT

Superabsorbents comprising at least one compound of formula (I)

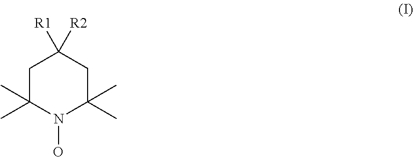

or salt thereof, where R1 and R2 independently are H, organic or functional substituents, exhibit improved stability against discolouration.

The present invention relates to a colour-stable superabsorbent, to a process for producing it and to the use thereof and to hygiene articles comprising it. A colour-stable superabsorbent is understood to mean a superabsorbent which is discoloured only to a minor degree, if at all, in the course of storage at elevated temperature and air humidity.

Superabsorbents are known. For such materials, names such as “highly swellable polymer”, “hydrogel” (often also used for the dry form), “hydrogel-forming polymer”, “water-absorbing polymer”, “absorbent gel-forming material”, “swellable resin”, “water-absorbing resin” or the like are also commonly used. These materials are crosslinked hydrophilic polymers, more particularly polymers formed from (co)polymerised hydrophilic monomers, graft (co)polymers of one or more hydrophilic monomers on a suitable graft base, crosslinked cellulose ethers or starch ethers, crosslinked carboxymethyl cellulose, partly crosslinked polyalkylene oxide or natural products swellable in aqueous liquids, for example guar derivatives, the most common being water-absorbing polymers based on partly neutralised acrylic acid. The essential properties of superabsorbents are their abilities to absorb several times their own weight of aqueous liquids and not to release the liquid again, even under a certain pressure. The superabsorbent, which is used in the form of a dry powder, is converted to a gel when it absorbs liquid, and specifically to a hydrogel when it absorbs water. Crosslinking is essential for synthetic superabsorbents and is an important difference from simple thickeners since it leads to the insolubility of the polymers in water. Soluble substances would be unusable as superabsorbents. By far the most important field of use of superabsorbents is the absorption of body fluids. Superabsorbents are used, for example, in diapers for infants, incontinence products for adults or feminine hygiene products. Other fields of use are, for example, as water-retaining agents in market gardening, as water stores for protection from fire, for liquid absorption in food packaging, or quite generally for absorbing moisture.

Superabsorbents are capable of absorbing several times their own weight of water and of retaining it under a certain pressure. In general, such a superabsorbent has a CRC (“centrifuge retention capacity”, see below for test method) of at least 5 g/g, preferably at least 10 g/g and more preferably at least 15 g/g. A “superabsorbent” may also be a mixture of different individual superabsorbent substances or a mixture of components which exhibit superabsorbent properties only when they interact (an example of this is a mixture of two polymers, one bearing anionic and the other cationic functional groups or groups that are converted into anionic and cationic groups upon interaction of the polymers); it is not so much the substance composition as the superabsorbent properties that are important here.

Important features for a superabsorbent are not only its absorption capacity, but also the ability to retain fluid under pressure (retention, usually expressed as “Absorption under Load” (“AUL”) or “Absorption against Pressure” (“AAP”), for test method see below) and the permeability, i.e. the ability to conduct fluid in the swollen state (usually expressed as “Saline Flow Conductivity” (“SFC”), for test method see below). Swollen gel can hinder or prevent fluid conductivity to as yet unswollen superabsorbent (“gel blocking”). Good conductivity properties for fluids are possessed, for example, by hydrogels which have a high gel strength in the swollen state. Gels with only a low gel strength are deformable under an applied pressure, in particular, the pressure applied by a human sitting while wearing a hygiene product comprising superabsorbent. This blocks pores in layers of such superabsorbents or superabsorbents/cellulose fibre mixtures that form the absorption core of a hygiene product and thus prevents fluid conductivity to as yet unswollen or incompletely swollen superabsorbent and fluid absorption by this as yet unswollen or incompletely swollen superabsorbent. An increased gel strength is generally achieved through a higher degree of crosslinking, but this reduces the absorption capacity of the product. An elegant method of increasing the gel strength is that of increasing the degree of crosslinking at the surface of the superabsorbent particles compared to the interior of the particles. To this end, superabsorbent particles which have a uniform crosslinking density are subjected to additional crosslinking in a thin surface layer of the particles thereof. This “surface postcrosslinking” or “surface crosslinking” increases the crosslinking density in the shell of the superabsorbent particles, which raises the absorption under compressive stress to a higher level. While the absorption capacity in the surface layer of the superabsorbent particles falls, their core retains its higher absorption capacity compared to the shell, such that the shell structure ensures improved permeability without occurrence of gel blocking without sacrificing too much overall absorption capacity. It is likewise known to achieve a similar non-uniform crosslinking density in superabsorbent particles by first producing superabsorbents which are relatively highly cross-linked overall and then removing crosslinks in the interior of the particles.

Processes for producing superabsorbents are also known. Superabsorbents based on acrylic acid, which are the most common on the market, are produced by free-radical polymerisation of acrylic acid in the presence of a crosslinker (the “inner crosslinker” or “internal crosslinker”), the acrylic acid being neutralised to a certain degree before, after or partly before and partly after the polymerisation, typically by adding alkali, usually an aqueous sodium hydroxide solution. The polymer gel thus obtained is comminuted (according to the polymerisation reactor used, this can be done simultaneously with the polymerisation) and dried. The dry powder thus obtained (the “base polymer”) is typically postcrosslinked on the surface of the particles, by reacting it with further crosslinkers, for instance organic crosslinkers or polyvalent cations, for example aluminium (usually used in the form of aluminium sulphate) or both, in order to obtain a more highly crosslinked surface layer compared to the particle interior.

A problem which often occurs in the case of superabsorbents is discolouration, which occurs in the course of storage under elevated temperature or elevated air humidity. Such conditions often occur in the case of storage of superabsorbents in tropical or subtropical countries. Superabsorbents tend to yellow under such conditions; they may even assume a brown or even almost black colour. This discolouration of the actually colourless superabsorbent powder is unsightly and undesired, since it is visible especially in the desired thin hygiene products, and consumers reject unsightly hygiene products. The cause of the discolouration has not been entirely clarified, but reactive compounds such as residual monomers from the polymerisation, the use of some initiators, impurities in the monomer or the neutralising agent, surface postcross-linkers or stabilisers in the monomers used appear to be involved. Generally, exposure of superabsorbent to light is not an issue in its discolouration as superabsorbents or superabsorbent-containing products such as diapers are stored in closed containers or storage facilities with no exposure to rain and therefore typically also not to sunlight.

Fredric L. Buchholz and Andrew T. Graham (editors) give, in: “Modern Superabsorbent Polymer Technology”, J. Wiley & Sons, New York, U.S.A./Wiley-VCH, Weinheim, Germany, 1997, ISBN 0-471-19411-5, a comprehensive review of superabsorbents, the properties thereof and processes for producing superabsorbents.

A number of methods to combat discolouration of superabsorbents are known from the prior art. WO 00/55 245 A1 teaches the stabilisation of superabsorbents against discolouration by treatment with an inorganic reducing agent and optionally a metal salt, for instance an alkaline earth metal salt, which is added after the polymerisation. The inorganic reducing agent is typically a hypophosphite, phosphite, bisulphite or sulphite. The metal salt is typically a colourless (the property “colourless” is often also simply referred to as “white”) phosphate, acetate or lactate, but not a halide. According to the teaching of WO 2006/058 682 A1, discolouration of super-absorbents is avoided when the drying and the postcrosslinking reaction are carried out in an atmosphere which is essentially free of oxidising gases. WO 2009/060 062 A1 or WO 2010/012 762 A2 teach the addition of sulphinic acid derivatives to superabsorbents in order to stabilise them against discolouration. EP 1 199 315 A2 teaches the use of a redox initiator system for initiating a polymerisation reaction, said redox initiator system comprising, as the reducing component, a sulphinic acid or a sulphinate, especially 2-hydroxysulphinatoacetic acid or a salt thereof. WO 99/18 067 A1 discloses particular hydroxyl- or aminoalkyl- or arylsulphinic acid derivatives or mixtures thereof and the use thereof as reducing agents which do not release formaldehyde. WO 2004/084 962 A1 relates to the use of these sulphinic acid derivatives as the reducing component of a redox initiator for polymerisation of partly neutralised acrylic acid to superabsorbents. Using phosphorus-containing compounds is also known: On example is Japanese patent application publication JP 05/086 251 that teaches the use of phosphoric acid derivatives or salts thereof, especially (1-hydroxyethane-1,1-diyl)bisphosphonic acid (also “1-hydroxyethylidene-1,1-diphosphonic acid”, “1-hydroxyethane-(1,1-diphosphonic acid)”, trivial name “etidronic acid”), ethylenediaminetetra(methylenephosphonic acid), diethylenetriamine-penta(methylenephosphonic acid) or the alkali metal or ammonium salts thereof as stabilisers of superabsorbents against discolouration. US 2005/0 085 604 A1 discloses the addition of chelating agents and oxidising or reducing agents to superabsorbents, the chelating agents also including those containing phosphorus.

Compounds comprising the 2,2,6,6-tetramethylpiperidino moiety are known for several applications in connection with acrylic acid, as well as in connection with superabsorbents.

U.S. Pat. No. 7,772,420 B2 discloses (meth)acrylic esters of monoalkoxylated polyols and their production. Such compounds are of use as internal crosslinkers for superabsorbents. Polymerisation inhibitors are added during their synthesis to avoid their premature polymerisation. Examples of polymerisation inhibitors include 4-hydroxy-2,2,6,6-tetramethylpiperidine N-oxyl, 4-oxo-2,2,6,6-tetramethylpiperidine N-oxyl, 4-acetoxy-2,2,6,6-tetramethylpiperidine N-oxyl, 2,2,6,6-tetra-methylpiperidine N-oxyl. U.S. Pat. No. 7,307,132 B2 teaches low-odour superabsorbents prepared from acrylic acid containing only low amounts of acetic or propionic acid as impurities. Common stabilisers of acrylic acid, in particular phenothiazine (dibenzene-1,4-thiazine) and 4-hydroxy-2,2,6,6-tetramethylpiperidine 1-oxyl are named as typical impurities. U.S. Pat. No. 7,038,081 B2 relates to heat integration of an acrylic acid and a polyacrylic acid plant, such as a superabsorbents plant. 4-hydroxy-2,2,6,6-tetramethylpiperidine is comprised in a list of polymerisation stabilisers for acrylic acid. U.S. Pat. No. 5,928,558 B2 and U.S. Pat. No. 6,080,864 B2 list a range of 2,2,6,6-tetramethylpiperidine N-oxyl derivatives as inhibitors to prevent premature polymerisation of acrylic acid and acrylic acid derivatives.

WO 2008/027488 A2, US 2010/0 063 469 A1 and WO 2004/018006 A1 list trimethylhexamethylene diisocyanate blocked with 2,2,3,6-tetramethylpiperidine-4-one [sic] as compound that may be used as surface postcrosslinker for superabsorbents.

U.S. Pat. No. 8,287,999 B2 and US 2008/0 124 551 A1 disclose superabsorbents that are coated with an elastic film-forming polymer by spray-coating and heat treating. During either one of these steps, an antioxidant is added to reduce or suppress degradation of the film-forming polymer by oxidative stress during heat treating or subsequent extended storage. 2,2,6,6-tetramethylpiperidine-4-one and 2,2,6,6-tetramethylpiperidin-4-ol are disclosed as antioxidants.

U.S. Pat. No. 9,162,007 B2 teaches a composite including a superabsorbent and cellulosic nanofibrils. The cellulosic nanofibrils are taught to be prepared, with reference to WO 2009/069 641 A1, by oxidising cellulose with sodium hypochlorite while using 2,2,6,6-tetramethylpiperidine-1-oxyl as an oxidation catalyst. Similarly, US 2012/0 302 440 A1 discloses a freeze-dried porous foam composite including a superabsorbent and cellulosic nanofibrils prepared by oxidising cellulose while using an oxidation catalyst that may be 2,2,6,6-tetramethylpiperidin-1-oxyl, 2,2,5,5,-tetramethylpyrrolidine-N-oxyl, 4-hydroxy-2,2,6,6-tetramethylpiperidin-1-oxyl or 4-acetamido-2,2,6,6-tetramethylpiperidin-1-oxyl.

CN 104 193 882 A relates to acrylate foams and their preparation by reverse suspension polymerisation. (2,2,6,6-tetramethylpiperidin-1-yl)oxidanyl is used as polymerisation initiator. WO 2015/020 512 A1 relates to polymer network materials prepared by reversible-deactivation radical polymerisation in compressed fluids. Polymerisation takes place in the presence of a controlling agent that may be 2,2,6,6-tetramethylpiperidine-1-oxyl.

The 2,2,6,6-tetramethylpiperidino moiety is generally known as bulky substituent and used in a number of other applications where steric hindrance may be of importance.

U.S. Pat. No. 7,211,622 B2 relates to aqueous resin compositions, in particular for use as coatings, where any publicly known acrylic monomer may be used as base monomer. Among those, polymerisable monomers containing piperidinic groups that are photostable in such as polymerisation are listed, in particular 4-(meth)acryloyloxy-2,2,6,6-tetramethylpiperidine, 4-(meth)acryloylamino-2,2,6,6-tetramethylpiperidine, 4-(meth)acryloyloxy-1,2,2,6,6-pentamethylpiperidine, 4-(meth)acryloyloxy-1-methoxy-2,2,6,6-tetramethylpiperidine, 4-cyano-4-(meth)acryloyloxy-2,2,6,6-tetramethylpiperidine, 1-(meth)acryloyl-4-(meth)acryloylamino-2,2,6,6-tetramethylpiperidine, and 4-crotonylamino-2,2,6,6-tetramethylpiperidine. US 2008/0 214 694 A1 also lists these compounds as monomers for producing waterborne curable resins. US 2011/0 017 611 A1 discloses oxygen-scavenging mixtures that comprise a nano-sized oxidisable metal, an electrolyte component and a non-electrolytic acidifying component. The mixture may further comprise a conventional additive such as antioxidants, UV absorbers and/or further light stabilisers. The list of such additives contains sterically hindered amines, a range of which bear the 2,2,6,6-tetramethylpiperidino moiety. EP 718 357 A2 discloses flame-retardant polymers having improved light stability and also discloses sterically hindered amines, including some bearing the 2,2,6,6-tetramethylpiperidino moiety, as light stabilisers. EP 548 901 A1 lists succinic acid dimethyl-1-(2-hydroxyethyl)-4-hydroxy-2,2,6,6-tetramethylpiperidine polycondensate among UV absorbents that may be comprised in microcapsules for use in agriculture.

It is a constant objective to find superabsorbents which are stabilised even better against discolouration, especially to yellowing or browning in the course of storage under elevated temperature and/or elevated air humidity and processes for production thereof. Further objects of the invention are uses of this superabsorbent, such as hygiene products comprising this super-absorbent and processes for production thereof.

This object was achieved by a superabsorbent comprising at least one compound of the formula (I):

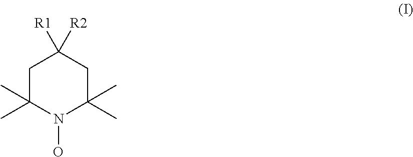

or salt thereof, where R1 and R2 independently are H, organic or functional substituents.

We have also found a process for producing the superabsorbent of the invention, namely by adding at least one compound of formula (I) to the superabsorbent. We have additionally found articles for absorption of fluids, especially hygiene articles for absorption of fluid excretions or fluid components of excretions, which comprise the superabsorbent of the invention. We have also found processes for production of such articles for absorption of fluids, the production of these articles involving addition of the superabsorbent of the invention thereto.

The inventive superabsorbents, surprisingly, exhibit good stability against discolouration without any notice of significant impairment in their service properties such as CRC, AUL or SFC.

R1 and R2 are independently hydrogen, organic or functional substituents. Examples of organic substituents are alkyl substituents, such as alkyl substituents having one to eight carbon atoms. Examples thereof are methyl, ethyl, n-propyl and iso-propyl. Examples of functional substituents are hydroxy, ether, ester or amido groups that in turn bear organic or functional substituents as defined here. Specific examples of substituents are acetyl or acetamido. R1 and R2 together also may form an oxo substituent, in other words may also form a keto group together with the carbon atom that bears them.

Further examples of compounds of formula (I) are the compounds of formulae (II), (III), (IV), (V) and (VI), in which R1 is H:

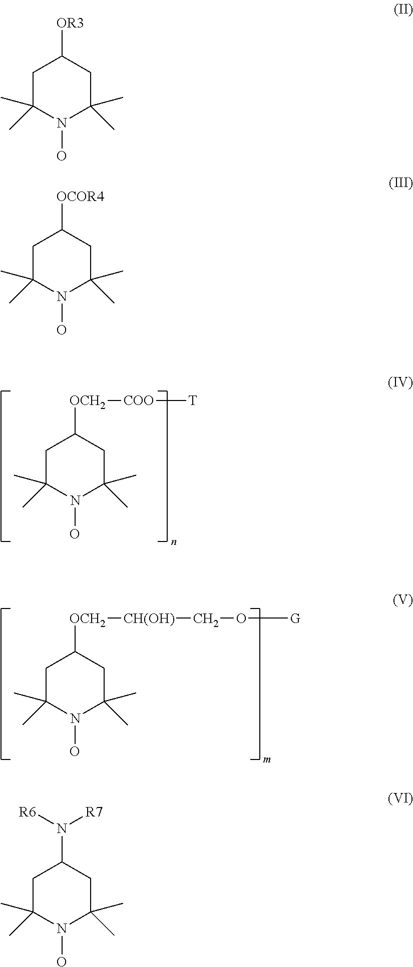

where R3 is alkenyl of 2 to 4 carbon atoms, propargyl, glycidyl, alkyl of 2 to 6 carbon atoms interrupted by one or two oxygen atoms, substituted by one to three hydroxyl groups or both interrupted by said oxygen atoms and substituted by said hydroxyl groups, or R3 is alkyl of 1 to 4 carbon atoms substituted by carboxy or by the alkali metal, ammonium or lower alkylammonium salts thereof; or R3 is alkyl substituted by —COOE where E is methyl or ethyl,

R4 is alkyl of 3 to 5 carbon atoms interrupted by —COO— or by —CO, or R4 is —CH₂(OCH₂CH₂)ₚOCH₃where p is 1 to 4; or R4 is —NHR5 where R5 is alkyl of 1 to 4 carbon atoms,

n is 2 to 4,

when n is 2, T is —(CH₂CHR—O)_{q}CH₂CHR—, where q is 0 or 1, and R is hydrogen or methyl,

when n is 3, T is glyceryl,

when n is 4, T is neopentanetetrayl,

m is 2 or 3,

when m is 2, G is —(CH₂CHR—O)ᵣCH₂CHR—, where r is 0 to 3, and R is hydrogen or methyl,

when m is 3, G is glyceryl,

R6 is hydrogen, alkyl of 1 to 4 carbon atoms, or said alkyl substituted by one or two hydroxyl, interrupted by one or two oxygen atoms, or both substituted by one hydroxyl and interrupted by one or two oxygen atoms,

R7 is —CO—R8 where R8 has the same meaning as R6, or R8 is —NHR9 wherein R9 is alkyl of 1 to 4 carbon atoms, said alkyl substituted by one or two hydroxyl, substituted by alkoxy of 1 to 2 carbon atoms, or said alkyl both substituted by one hydroxyl and by one alkoxy of 1 to 2 carbon atoms, or

R6 and R7 together are —CO—CH₂ CH₂—CO—, —CO—CH═CH—CO— or —(CH₂)₆—CO—; and

with the proviso that, when R8 is alkyl of 1 to 4 carbon atoms, R6 is not hydrogen.

Preferably, R3 is allyl, methallyl, glycidyl, 2,3-dihydroxypropyl, 2-hydroxy-4-oxapentyl or —CH₂COOH.

Preferably R4 is methoxymethyl, 2-methoxyethoxymethyl, 2-(2-methoxyethoxy) ethoxymethyl, —CH₂COCH₃, —CH₂CH₂COOCH₃ or butylamino.

Preferably, n is 2, T is —(CH₂CHR—O)_{q}CH₂CHR—, where q is 0, and R is hydrogen.

Preferably, m is 2, G is —(CH₂CHR—O)ᵣCH₂CHR—, where r is 0 or 1, and R is hydrogen.

Preferably, R6 is hydrogen or n-butyl.

Preferably R7 is —CO—R8 where R8 is hydrogen, methyl, ethyl, n-propyl, isopropyl, methoxymethyl or 2-methoxyethoxymethyl; or R7 is N-butylcarbamoyl.

These compounds are further described in U.S. Pat. No. 6,080,864 and U.S. Pat. No. 5,928,558, both patents expressly referred to herein and incorporated by reference.

The compounds of formula (I) may be used in the form of a salt thereof. Such salt may easily be formed by reaction of a compound of formula (I) with an acid such as a carboxylic acid.

One specific preferred example of a compound of formula (I) is (2,2,6,6-Tetramethylpiperidin-1-yl)oxyl, also named (2,2,6,6-tetramethylpiperidin-1-yl)oxidanyl. This compound corresponds to formula (I) where R1=R2=H and is often referred to as “TEMPO”. Other specific examples of preferred compounds of formula (I) or salts thereof are 4-Hydroxi-(2,2,6,6-tetramethylpiperidin-1-yl)oxyl (“4-Hydroxy-TEMPO”, R1=H, R2=OH) and 4-Acetamido-(2,2,6,6-tetramethylpiperidin-1-yl)oxyl (“4-Acetamido-TEMPO”, R1=H, R2=N(H)C(O)CH₃), 4-Acetyl-(2,2,6,6-tetramethylpiperidin-1-yl)oxyl (“4-Acetyl-TEMPO”, R1=H, R2=OC(O)CH3), 4-Oxo-(2,2,6,6-Tetramethylpiperidin-1-yl)oxyl (R1 together with R2 is an oxo substituent) and 1,4-dihydroxy-2,2,6,6-tetramethylpiperidinium 2-hydroxy-1,2,3-propanetricarboxylate (in other words, the salt of 4-Hydroxi-TEMPO with citric acid).

These compounds are known and are commercially available. Synthesis routes to these compounds are therefore also known. Generally, these compounds are produced by methods involving condensation reactions of acetone and ammonia and subsequent oxidation. These compounds are stable radicals and are of use as reactants, markers, catalysts or mediators in a range of organic synthesis reactions.

The superabsorbent comprises generally at least 0.0001 wt. % of the compound of formula (I), preferably at least 0.0005 wt. % and more preferably 0.001 wt. % and generally at most 5 wt. %, preferably at most 1 wt. % and more preferably at most 1 wt. % based on the total weight of the dry superabsorbent comprising the compound of formula (I). “Dry” means a moisture content of not more than 5 wt. %. Commercial superabsorbents typically have a moisture content of 1 to 5 wt. %. If the superabsorbent comprises more than one compound of formula (I), these figures relate to the total amount of compounds if formula (I) in the superabsorbent.

The superabsorbent if the inventions are prepared by adding at least one compound of formula (I) to a superabsorbent. Methods and mixers for adding additives to a pulverulent product, as is done by adding compound of formula (I) to a superabsorbent, are known. Preferably, mixing is carried out in mixers with moving mixing tools, such as screw mixers, disk mixers, paddle mixers or shovel mixers, or mixers with other mixing tools.

It is important that the compound of formula (I) is added to a superabsorbent. Adding the compound of formula (I) to the monomer solution used for producing a superabsorbent will not lead to a superabsorbent comprising the compound of formula (I) since the compound of formula (I) will be consumed during the polymerisation step to produce the superabsorbent.

The superabsorbent that is to be admixed with compound of formula (I) is any known super-absorbent. To produce the superabsorbent of the invention, at least one compound of formula (I) is mixed with the superabsorbent.

Preferably, the superabsorbent that is to be admixed with the compound of formula (I) is a superabsorbent on the basis of partially neutralised crosslinked polyacrylic acid. Such super-absorbents are prepared by polymerising an aqueous monomer solution comprising
a) at least one ethylenically unsaturated monomer which bears acid groups and is optionally present at least partly in salt form,
b) at least one crosslinker,
c) at least one initiator,
d) optionally one or more ethylenically unsaturated monomers copolymerisable with the monomers mentioned under a),
e) optionally one or more water-soluble polymers,

the process further comprising

drying of the resulting polymer,

optionally grinding of the dried polymer and sieving of the ground polymer and

optionally surface postcrosslinking of the dried and optionally ground and sieved polymer.

In the case of superabsorbents produced according to the process referred to directly above, the compound of formula (I) is added after the polymerisation step, preferably after the drying step, more preferably during or after the grinding and sieving step and, if the superabsorbent is also surface postcrosslinked, the compound of formula (I) is most preferably added after the surface postcrosslinking step. Most preferably, the superabsorbent is surface postcrosslinked and mixed with the compound of formula (I) after the surface postcrosslinking step. One convenient point to add compounds of formula (I) is a cooler downstream of the heater used to conduct the surface postcrosslinking reaction. Compounds of formula (I) may be added in the same step, and even together with, other additives that are added to the superabsorbent, for example dedusting agents, surface complexing agents, caking prevention agents, or any other additives or auxiliaries.

The monomers a) are preferably water-soluble, i.e. the solubility in water at 23° C. is typically at least 1 g/100 g of water, preferably at least 5 g/100 g of water, more preferably at least 25 g/100 g of water and most preferably at least 35 g/100 g of water.

Suitable monomers a) are, for example, ethylenically unsaturated carboxylic acids or salts thereof, such as acrylic acid, methacrylic acid, maleic acid or salts thereof, maleic anhydride and itaconic acid or salts thereof. Particularly preferred monomers are acrylic acid and methacrylic acid. Very particular preference is given to acrylic acid.

Further suitable monomers a) are, for example, ethylenically unsaturated sulphonic acids, such as styrenesulphonic acid and 2-acrylamido-2-methylpropanesulphonic acid (AMPS).

Impurities can have a considerable influence on the polymerisation. The raw materials used should therefore have a maximum purity. It is therefore often advantageous to specially purify the monomers a). Suitable purification processes are described, for example, in WO 2002/055469 A1, WO 2003/078378 A1 and WO 2004/035514 A1. A suitable monomer a) is, for example, acrylic acid purified according to WO 2004/035514 A1 and comprising 99.8460% by weight of acrylic acid, 0.0950% by weight of acetic acid, 0.0332% by weight of water, 0.0203% by weight of propionic acid, 0.0001% by weight of furfurals, 0.0001% by weight of maleic anhydride, 0.0003% by weight of diacrylic acid and 0.0050% by weight of hydroquinone monomethyl ether.

The proportion of acrylic acid and/or salts thereof in the total amount of monomers a) is preferably at least 50 mol %, more preferably at least 90 mol %, most preferably at least 95 mol %.

The monomer solution comprises preferably at most 250 ppm by weight, preferably at most 130 ppm by weight, more preferably at most 70 ppm by weight and preferably at least 10 ppm by weight, more preferably at least 30 ppm by weight, especially around 50 ppm by weight, of hydroquinone monoether, based in each case on the unneutralised monomer a); neutralised monomer a), i.e. a salt of the monomer a), is considered for arithmetic purposes to be unneutralised monomer. For example, the monomer solution can be prepared by using an ethylenically unsaturated monomer bearing acid groups with an appropriate content of hydroquinone monoether.

Preferred hydroquinone monoethers are hydroquinone monomethyl ether (MEHQ) and/or alpha-tocopherol (vitamin E).

Suitable crosslinkers b) are compounds having at least two groups suitable for crosslinking. Such groups are, for example, ethylenically unsaturated groups which can be polymerised free-radically into the polymer chain, and functional groups which can form covalent bonds with the acid groups of the monomer a). In addition, polyvalent metal salts which can form coordinate bonds with at least two acid groups of the monomer a) are also suitable as crosslinkers b).

Crosslinkers b) are preferably compounds having at least two polymerisable groups which can be polymerised free-radically into the polymer network. Suitable crosslinkers b) are, for example, ethylene glycol dimethacrylate, diethylene glycol diacrylate, polyethylene glycol diacrylate, allyl methacrylate, trimethylolpropane triacrylate, triallylamine, tetraallylammonium chloride, tetraallyloxyethane, as described in EP 530 438 A1, di- and triacrylates, as described in EP 547 847 A1, EP 559 476 A1, EP 632 068 A1, WO 93/21237 A1, WO 2003/104299 A1, WO 2003/104300 A1, WO 2003/104301 A1 and DE 103 31 450 A1, mixed acrylates which, as well as acrylate groups, comprise further ethylenically unsaturated groups, as described in DE 103 31 456 A1 and DE 103 55 401 A1, or crosslinker mixtures, as described, for example, in DE 195 43 368 A1, DE 196 46 484 A1, WO 90/15830 A1 and WO 2002/32962 A2.

Preferred crosslinkers b) are pentaerythrityl triallyl ether, tetraallyloxyethane, methylenebismethacrylamide, 15- to 20-tuply ethoxylated trimethylolpropane triacrylate, 15-20-tuply ethoxylated glyceryl triacrylate, polyethylene glycol diacrylate having between 4 and 45—CH2CH2O units in the molecule chain, trimethylolpropane triacrylate and triallylamine.

Very particularly preferred crosslinkers b) are the polyethoxylated and/or -propoxylated glycerols which have been esterified with acrylic acid or methacrylic acid to give di- or triacrylates, as described, for example, in WO 2003/104301 A1. Di- and/or triacrylates of 3- to 10-tuply ethoxylated glycerol are particularly advantageous. Very particular preference is given to di- or triacrylates of 1- to 5-tuply ethoxylated and/or propoxylated glycerol. Most preferred are the triacrylates of 3- to 5-tuply ethoxylated and/or propoxylated glycerol, especially the triacrylate of 3-tuply ethoxylated glycerol.

The amount of crosslinker b) is preferably 0.05 to 1.5% by weight, more preferably 0.1 to 1% by weight and most preferably 0.3 to 0.6% by weight, based in each case on monomer a). With rising crosslinker content, the centrifuge retention capacity (CRC) falls and the absorption under a pressure of 0.3 psi (AUL0.3psi) rises.

The initiators c) used may be all compounds which generate free radicals under the polymerisation conditions, for example thermal initiators, redox initiators and/or photoinitiators. Suitable redox initiators are sodium peroxodisulphate/ascorbic acid, hydrogen peroxide/ascorbic acid, sodium peroxodisulphate/sodium bisulphite and hydrogen peroxide/sodium bisulphite. Preference is given to using mixtures of thermal initiators and redox initiators, such as sodium peroxodisulphate/hydrogen peroxide/ascorbic acid. The reducing component used is preferably a sulphonic acid derivative, in particular 2-hydroxy-2-sulphonatoacetic acid and salts thereof, especially the sodium salts thereof, and among these especially the disodium salt thereof, and mixtures therewith (for example those mixtures with the corresponding sulphite that are available from L. Bruggemann K G, Salzstrasse 131, 74076 Heilbronn, Germany, under the BRUGGOLIT® FF6M or BRUGGOLIT® FF7 marks, or alternatively BRUGGOLITE® FF6M or BRUGGOLITE® FF7). The initiators are, incidentally, used in customary amounts. The customary amount of the reducing component of a redox initiator is generally at least 0.00001% by weight, preferably at least 0.0001% by weight and more preferably at least 0.001% by weight, and generally at most 0.2% by weight and preferably at most 0.1% by weight, based in each case on the amount of monomers a) and d). When, however, the sole reducing component used in the redox initiator is sulphonic acid derivative, the added amount thereof is generally at least 0.001% by weight, preferably at least 0.01% by weight and more preferably at least 0.03% by weight, and generally at most 1.0% by weight, preferably at most 0.3% by weight and more preferably at most 0.2% by weight, based in each case on the amount of monomers a) and d). The customary amount of the oxidising component of a redox initiator is generally 0.0001% by weight and more preferably at least 0.001% by weight, and generally at most 2% by weight and preferably at most 1.0% by weight, based in each case on the amount of monomers a) and d). The customary amount of the thermal initiators is generally 0.01% by weight and more preferably at least 0.1% by weight, and generally at most 2% by weight and preferably at most 1.0% by weight, based in each case on the amount of monomers a) and d). The customary amount of the photoinitiators is generally 0.001% by weight and more preferably at least 0.01% by weight, and generally at most 1.0% by weight and preferably at most 0.2% by weight, based in each case on the amount of monomers a) and d).

Ethylenically unsaturated monomers d) copolymerisable with the ethylenically unsaturated monomers a) bearing acid groups are, for example, acrylamide, methacrylamide, hydroxyethyl acrylate, hydroxyethyl methacrylate, dimethylaminoethyl methacrylate, dimethylaminoethyl acrylate, dimethylaminopropyl acrylate, diethylaminopropyl acrylate, dimethylaminoethyl methacrylate, diethylaminoethyl methacrylate, maleic acid or salts thereof and maleic anhydride.

The water-soluble polymers e) used may be polyvinyl alcohol, polyvinylpyrrolidone, starch, starch derivatives, modified cellulose, such as methylcellulose or hydroxyethylcellulose, gelatin, polyglycols or polyacrylic acids, preferably starch, starch derivatives and modified cellulose.

Typically, an aqueous monomer solution is used. The water content of the monomer solution is preferably from 40 to 75% by weight, more preferably from 45 to 70% by weight and most preferably from 50 to 65% by weight. It is also possible to use monomer suspensions, i.e. oversaturated monomer solutions. With rising water content, the energy requirement in the subsequent drying rises, and, with falling water content, the heat of polymerisation can only be removed inadequately.

For optimal action, the preferred polymerisation inhibitors require dissolved oxygen. The monomer solution can therefore be freed of dissolved oxygen before the polymerisation by inertisation, i.e. flowing an inert gas through, preferably nitrogen or carbon dioxide. The oxygen content of the monomer solution is preferably lowered before the polymerisation to less than 1 ppm by weight, more preferably to less than 0.5 ppm by weight, most preferably to less than 0.1 ppm by weight.

The monomer mixture may comprise further components. Examples of further components used in such monomer mixtures are, for instance, chelating agents in order to keep metal ions in solution, or inorganic powders in order to increase the stiffness of the superabsorbent in the swollen state, or recycled undersize from a later grinding operation. It is possible here to use all known additions to the monomer mixture. Even though only “solution” is discussed here in connection with the monomer mixture, this also means the polymerisation of a suspension, for instance when insoluble constituents are added to the monomer mixture.

The acid groups of the polymer gels resulting from the polymerisation have typically been partly neutralised. Neutralisation is preferably carried out at the monomer stage; in other words, salts of the monomers bearing acid groups or, to be precise, a mixture of monomers bearing acid groups and salts of the monomers bearing acid groups (“partly neutralised acid”) are used as component a) in the polymerisation. This is typically accomplished by mixing the neutralising agent as an aqueous solution or preferably also as a solid into the monomer mixture intended for polymerisation or preferably into the monomer bearing acid groups or a solution thereof. The degree of neutralisation is preferably from 25 to 95 mol %, more preferably from 50 to 80 mol % and most preferably from 65 to 72 mol %, for which the customary neutralising agents can be used, preferably alkali metal hydroxides, alkali metal oxides, alkali metal carbonates or alkali metal hydrogencarbonates and also mixtures thereof. Instead of alkali metal salts, it is also possible to use ammonium salts. Particularly preferred alkali metals are sodium and potassium, but very particular preference is given to sodium hydroxide, sodium carbonate or sodium hydrogen-carbonate and also mixtures thereof.

However, it is also possible to carry out neutralisation after the polymerisation, at the stage of the polymer gel formed in the polymerisation. It is also possible to neutralise up to 40 mol %, preferably 10 to 30 mol % and more preferably 15 to 25 mol % of the acid groups before the polymerisation by adding a portion of the neutralising agent directly to the monomer solution and setting the desired final degree of neutralisation only after the polymerisation, at the polymer gel stage. When the polymer gel is neutralised at least partly after the polymerisation, the polymer gel is preferably comminuted mechanically, for example by means of an extruder, in which case the neutralising agent can be sprayed, sprinkled or poured on and then carefully mixed in. To this end, the gel mass obtained can be repeatedly extruded for homogenisation.

However, preference is given to performing the neutralisation at the monomer stage. In other words: in a very particularly preferred embodiment, the monomer a) used is a mixture of 25 to 95 mol %, more preferably from 50 to 80 mol % and most preferably from 65 to 75 mol % of salt of the monomer bearing acid groups, and the remainder to 100 mol % of monomer bearing acid groups. This mixture is, for example, a mixture of sodium acrylate and acrylic acid or a mixture of potassium acrylate and acrylic acid.

In a preferred embodiment, the neutralising agent used for the neutralisation is one whose iron content is generally below 10 ppm by weight, preferably below 2 ppm by weight and more preferably below 1 ppm by weight. Likewise desired is a low content of chloride and anions of oxygen acids of chlorine. A suitable neutralising agent is, for example, the 50% by weight sodium hydroxide solution or potassium hydroxide solution which is typically traded as “membrane grade”; even more pure and likewise suitable, but also more expensive, is the 50% by weight sodium hydroxide solution or potassium hydroxide solution typically traded as “amalgam grade” or “mercury process”.

Processes for production of superabsorbents from monomer mixtures, such as those described by way of example above, are known in principle. Suitable polymerisation reactors are, for example, kneading reactors or belt reactors. In the kneader, the polymer gel formed in the polymerisation of an aqueous monomer solution or suspension is comminuted continuously by, for example, contra-rotatory stirrer shafts, as described in WO 2001/38402 A1. Polymerisation on a belt is described, for example, in EP 955 086 A2, DE 38 25 366 A1 and U.S. Pat. No. 6,241,928. Polymerisation in a belt reactor forms, like the likewise known polymerisation in batchwise operation or in a tubular reactor, as described, for example, in EP 445 619 A2 and DE 19 846 413 A1, a polymer gel which has to be comminuted in a further process step, for example in a meat grinder, extruder or kneader. It is also possible to produce spherical or differently shaped superabsorbent particles by suspension or emulsion polymerisation, as described, for example, in EP 457 660 A1, or by spray or droplet polymerisation processes, as described, for example, in EP 348 180 A1, EP 816 383 A1, WO 96/404 27 A1, U.S. Pat. No. 4,020,256, US 2002/0 193 546 A1, DE 35 19 013 A1, DE 10 2005 044 035 A1, WO 2007/093531 A1, WO 2008/086 976 Al or WO 2009/027 356 A1. Likewise known are processes in which the monomer mixture is applied to a substrate, for example a nonwoven web, and polymerised, as described, for instance, in WO 02/94 328 A2 and WO 02/94 329 A1.

The polymer gel obtained from the aqueous solution polymerisation and optional subsequent neutralisation is then preferably dried with a belt drier until the residual moisture content is preferably 0.5 to 15% by weight, more preferably 1 to 10% by weight and most preferably from 2 to 8% by weight (see below for test method for the residual moisture or water content). In the case of too high a residual moisture content, the dried polymer gel has too low a glass transition temperature Tg and can be processed further only with difficulty. In the case of too low a residual moisture content, the dried polymer gel is too brittle and, in the subsequent comminution steps, undesirably large amounts of polymer particles with an excessively low particle size are obtained (“fines”). The solids content of the gel before drying is generally from 25 to 90% by weight, preferably from 30 to 80% by weight, more preferably from 35 to 70% by weight and most preferably from 40 to 60% by weight. Optionally, however, it is also possible to dry using a fluidised bed drier or a heatable mixer with a mechanical mixing unit, for example a paddle drier or a similar drier with mixing tools of different design. Optionally, the drier can be operated under nitrogen or another non-oxidising inert gas or at least under reduced partial oxygen pressure in order to prevent oxidative yellowing processes. In general, however, even sufficient venting and removal of water vapour leads to an acceptable product. In general, a minimum drying time is advantageous with regard to colour and product quality.

During the drying, the residual monomer content in the polymer particles is also reduced, and last residues of the initiator are destroyed.

Thereafter, the dried polymer gel is ground and classified, and the apparatus used for grinding may typically be single or multistage roll mills, preferably two- or three-stage roll mills, pin mills, hammer mills or vibratory mills. Oversize gel lumps which often still have not dried on the inside are elastomeric, lead to problems in the grinding and are preferably removed before the grinding, which can be done in a simple manner by wind sifting or by means of a sieve (“guard sieve” for the mill). In view of the mill used, the mesh size of the sieve should be selected such that a minimum level of disruption resulting from oversize, elastomeric particles occurs.

Excessively large, insufficiently finely ground superabsorbent particles are perceptible as coarse particles in their predominant use, in hygiene products such as diapers; they also lower the mean initial swell rate of the superabsorbent. Both are undesired. Advantageously, coarse-grain polymer particles are therefore removed from the product. This is done by conventional classification processes, for example wind sifting, or by sieving through a sieve with a mesh size of at most 1000 μm, preferably at most 900 μm, more preferably at most 850 μm and most preferably at most 800 μm. For example, sieves of mesh size 700 μm, 650 μm or 600 μm are used. The coarse polymer particles (“oversize”) removed may, for cost optimisation, be sent back to the grinding and sieving cycle or be processed further separately.

Polymer particles with too small a particle size lower the permeability (SFC). Advantageously, this classification therefore also removes fine polymer particles. This can, if sieving is effected, conveniently be done by means of a sieve of mesh size at most 300 μm, preferably at most 200 μm, more preferably at most 150 μm and most preferably at most 100 μm. The fine polymer particles (“undersize” or “fines”) removed can, for cost optimisation, be sent back as desired to the monomer stream, to the polymerising gel, or to the fully polymerised gel before the drying of the gel.

The mean particle size of the polymer particles removed as the product fraction is generally at least 200 μm, preferably at least 250 μm and more preferably at least 300 μm, and generally at most 600 μm and more preferably at most 500 μm. The proportion of particles with a particle size of at least 150 μm is generally at least 90% by weight, more preferably at least 95% by weight and most preferably at least 98% by weight. The proportion of particles with a particle size of at most 850 μm is generally at least 90% by weight, more preferably at least 95% by weight and most preferably at least 98% by weight.

In some other known production processes for superabsorbents, especially in the case of suspension polymerisation, spray or dropletisation polymerisation, the selection of the process parameters defines the particle size distribution. These processes directly give rise to particulate superabsorbents of the desired particle size, such that grinding and sieving steps can often be dispensed with. In some processes (especially in the case of spray or dropletisation polymerisation), a dedicated drying step can often also be dispensed with.

The polymer thus prepared has superabsorbent properties and is covered by the term “super-absorbent”. Its CRC is typically comparatively high, but its AUL or SFC comparatively low. A superabsorbent of this type, i.e. one that is not surface postcrosslinked, is often referred to as “base polymer” to distinguish it from a surface postcrosslinked superabsorbent produced therefrom.

The base polymer is optionally surface postcrosslinked.

Suitable postcrosslinkers are compounds which comprise groups which can form bonds with at least two functional groups of the superabsorbent particles. This is well-known technology. In the case of the acrylic acid/sodium acrylate-based superabsorbents prevalent on the market, suitable surface postcrosslinkers are compounds which comprise groups which can form bonds with at least two carboxylate groups, for example amide acetals or carbamates, polyhydric alcohols, cyclic carbonates, diglycidyl compounds such as ethylene glycol diglycidyl ether or bisoxazolines.

Preferred postcrosslinkers are 2-oxazolidones such as 2-oxazolidone and N-(2-hydroxyethyl)-2-oxazolidone, N-methyl-2-oxazolidone, N-acyl-2-oxazolidones such as N-acetyl-2-oxazolidone, 2-oxotetrahydro-1,3-oxazine, bicyclic amide acetals such as 5-methyl-1-aza-4,6-dioxabicyclo[3.3.0]octane, 1-aza-4,6-dioxabicyclo[3.3.0]octane and 5-isopropyl-1-aza-4,6-dioxabicyclo[3.3.0]octane, bis-2-oxazolidones, poly-2-oxazolidones and ethylene glycol diglycidyl ether.

Particularly preferred postcrosslinkers are 2-oxazolidone, N-methyl-2-oxazolidone, N-(2-hydroxyethyl)-2-oxazolidone and N-hydroxypropyl-2-oxazolidone.

It is also possible to use any mixtures of different postcrosslinkers.

The postcrosslinker is generally used in an amount of at least 0.001% by weight, preferably of at least 0.02% by weight, more preferably of at least 0.05% by weight, and generally at most 2% by weight, preferably at most 1% by weight, more preferably at most 0.3% by weight, for example at most 0.15% by weight or at most 0.095% by weight, based in each case on the mass of the base polymer contacted therewith (for example the sieve fraction in question).

The postcrosslinking is typically performed in such a way that a solution of the postcrosslinker is sprayed onto the dried base polymer particles. After the spray application, the polymer particles coated with postcrosslinker are dried thermally, and the postcrosslinking reaction can take place either before or during the drying. If surface postcrosslinkers with polymerisable groups are used, the surface postcrosslinking can also be effected by means of free-radically induced polymerisation of such groups by means of common free-radical formers or else by means of high-energy radiation, for example UV light. This can be done in parallel with or instead of the use of postcrosslinkers which form covalent or ionic bonds to functional groups at the surface of the base polymer particles.

The spray application of the postcrosslinker solution is preferably carried out in mixers with moving mixing tools, such as screw mixers, disk mixers, paddle mixers or shovel mixers, or mixers with other mixing tools. Particular preference is given, however, to vertical mixers. It is also possible to spray on the postcrosslinker solution in a fluidised bed. Suitable mixers are obtainable, for example, as Pflugschar® mixers from Gebr. Lodige Maschinenbau GmbH, Elsener-Strasse 7-9, 33102 Paderborn, Germany, or as Schugi® Flexomix® mixers, Vrieco-Nauta® mixers or Turbulizer® mixers from Hosokawa Micron BV, Gildenstraat 26, 7000 AB Doetinchem, the Netherlands.

The spray nozzles usable are not subject to any restriction. Suitable nozzles and atomisation systems are described, for example, in the following references: Zerstauben von Flüssigkeiten [Atomisation of Liquids], Expert-Verlag, vol. 660, Reihe Kontakt & Studium, Thomas Richter (2004) and in Zerstäubungstechnik [Atomisation Technology], Springer-Verlag, VDI-Reihe, Günter Wozniak (2002). It is possible to use mono- and polydisperse spray systems. Among the polydisperse systems, one-phase pressurised nozzles (jet- or lamella-forming), rotary atomisers, two-phase atomisers, ultrasound atomisers and impingement nozzles are suitable. In the case of the two-phase atomisers, the liquid phase can be mixed with the gas phase either internally or externally. The spray profile of the nozzles is uncritical and may assume any desired form, for example a round jet, flat jet, wide angle round beam or circular ring spray profile. It is advantageous to use a non-oxidising gas if two-phase atomisers are used, particular preference being given to nitrogen, argon or carbon dioxide. Such nozzles can be supplied with the liquid to be sprayed under pressure. The atomisation of the liquid to be sprayed can be effected by expanding it in the nozzle bore on attainment of a particular minimum velocity. In addition, it is also possible to use one-phase nozzles for the inventive purpose, for example slit nozzles or swirl chambers (full-cone nozzles) (for example from Düsen-Schlick GmbH, Germany, or from Spraying Systems Deutschland GmbH, Germany). Such nozzles are also described in EP 0 534 228 A1 and EP 1 191 051 A2.

The postcrosslinkers are typically used in the form of an aqueous solution. When exclusively water is used as the solvent, a surfactant or deagglomeration assistant is advantageously added to the postcrosslinker solution or actually to the base polymer. This improves the wetting behavior and reduces the tendency to form lumps.

All anionic, cationic, nonionic and amphoteric surfactants are suitable as deagglomeration assistants, but preference is given to nonionic and amphoteric surfactants for skin compatibility reasons. The surfactant may also comprise nitrogen. For example, sorbitan monoesters, such as sorbitan monococoate and sorbitan monolaurate, or ethoxylated variants thereof, for example Polysorbat 20®, are added. Further suitable deagglomeration assistants are the ethoxylated and alkoxylated derivatives of 2-propylheptanol, which are sold under the Lutensol XL® and Lutensol XP® brands (BASF SE, Carl-Bosch-Straβe 38, 67056 Ludwigshafen, Germany).

The deagglomeration assistant can be metered in separately or added to the postcrosslinker solution. Preference is given to simply adding the deagglomeration assistant to the postcrosslinker solution.

The amount of the deagglomeration assistant used, based on base polymer, is, for example, from 0 to 0.1% by weight, preferably from 0 to 0.01% by weight, more preferably from 0 to 0.002% by weight. The deagglomeration assistant is preferably metered in such that the surface tension of an aqueous extract of the swollen base polymer and/or of the swollen postcrosslinked water-absorbing polymer at 23° C. is at least 0.060 N/m, preferably at least 0.062 N/m, more preferably at least 0.065 N/m, and advantageously at most 0.072 N/m.

The aqueous postcrosslinker solution may, as well as the at least one postcrosslinker, also comprise a cosolvent. The penetration depth of the postcrosslinker into the polymer particles can be adjusted via the content of nonaqueous solvent and total amount of solvent. Industrially highly suitable cosolvents are C1-C6-alcohols such as methanol, ethanol, n-propanol, isopropanol, n-butanol, sec-butanol, tert-butanol or 2-methyl-1-propanol, C2-C5-diols such as ethylene glycol, 1,2-propylene glycol or 1,4-butanediol, ketones such as acetone, or carboxylic esters such as ethyl acetate. A disadvantage of some of these cosolvents is that they have typical intrinsic odors. The cosolvent itself is ideally not a postcrosslinker under the reaction conditions. However, it may arise in the boundary case and depending on the residence time and temperature that the cosolvent contributes partly to crosslinking. This is the case especially when the postcrosslinker is relatively slow to react and can therefore also constitute its own cosolvent, as is the case, for example, when cyclic carbonates, diols or polyols are used. Such postcrosslinkers can also be used in the function as a cosolvent in a mixture with more reactive postcrosslinkers, since the actual postcrosslinking reaction can then be performed at lower temperatures and/or with shorter residence times than in the absence of the more reactive crosslinker. Since the cosolvent is used in relatively large amounts and some also remains in the product, it must not be toxic. Diols, polyols and cyclic carbonates are also suitable as cosolvents. They fulfill this function in the presence of more reactive postcrosslinkers. Preferred cosolvents are, however, diols, especially when a reaction of the hydroxyl groups is hindered sterically by neighbouring groups. Although such diols are also suitable in principle as postcrosslinkers, this requires significantly higher reaction temperatures or optionally higher use amounts than for sterically unhindered diols. Particularly preferred combinations of low-reactivity postcrosslinker as a cosolvent and reactive postcrosslinker are combinations of polyhydric alcohols, diols and polyols, with amide acetals or carbamates. Suitable combinations are, for example, 2-oxazolidone/1,2-propanediol and N-(2-hydroxyethyl)-2-oxazolidone/1,2-propanediol, and also ethylene glycol diglycidyl ether/1,2-propanediol. Very particularly preferred combinations are 2-oxazolidone/1,3-propanediol and N-(2-hydroxyethyl)-2-oxazolidone/1,3-propanediol. Further preferred combinations are those with ethylene glycol diglycidyl ether or glyceryl di- or triglycidyl ether with the following solvents, cosolvents or cocrosslinkers: isopropanol, 1,3-propanediol, 1,2-propylene glycol or mixtures thereof. Further preferred combinations are those with 2-oxazolidone or (2-hydroxyethyl)-2-oxazolidone in the following solvents, cosolvents or cocrosslinkers: isopropanol, 1,3-propanediol, 1,2-propylene glycol, ethylene carbonate, propylene carbonate or mixtures thereof.

Frequently, the concentration of the cosolvent in the aqueous postcrosslinker solution is from 15 to 50% by weight, preferably from 15 to 40% by weight and more preferably from 20 to 35% by weight, based on the postcrosslinker solution. In the case of cosolvents which have only limited miscibility with water, the aqueous postcrosslinker solution will advantageously be adjusted such that only one phase is present, optionally by lowering the concentration of the cosolvent.

In a preferred embodiment, no cosolvent is used. The postcrosslinker is then employed only as a solution in water, optionally with addition of a deagglomeration assistant.

The concentration of the at least one postcrosslinker in the aqueous postcrosslinker solution is typically from 1 to 20% by weight, preferably from 1.5 to 10% by weight and more preferably from 2 to 5% by weight, based on the postcrosslinker solution.

The total amount of the postcrosslinker solution based on base polymer is typically from 0.3 to 15% by weight and preferably from 2 to 6% by weight.

The actual surface postcrosslinking by reaction of the surface postcrosslinker with functional groups at the surface of the base polymer particles is usually carried out by heating the base polymer wetted with surface postcrosslinker solution, typically referred to as “drying” (but not to be confused with the above-described drying of the polymer gel from the polymerisation, in which typically very much more liquid has to be removed). The drying can be effected in the mixer itself, by heating the jacket, by means of heat exchange surfaces or by blowing in warm gases. Simultaneous admixing of the superabsorbent with surface postcrosslinker and drying can be effected, for example, in a fluidised bed drier. The drying is, however, usually carried out in a downstream drier, for example a tray drier, a rotary tube oven, a paddle or disk drier or a heatable screw. Suitable driers are obtainable, for example, as Solidair® or Torusdisc® driers from Bepex International LLC, 333 N.E. Taft Street, Minneapolis, Minn. 55413, U.S.A., or as paddle or shovel driers or else as fluidised bed driers from Nara Machinery Co., Ltd., European Office, Europaallee 46, 50226 Frechen, Germany.

It is possible to heat the polymer particles by means of contact surfaces in a downstream drier for the purpose of drying and performing the surface postcrosslinking, or by means of warm inert gas supply, or by means of a mixture of one or more inert gases with steam, or only with steam alone. In the case of supply of the heat by means of contact surfaces, it is possible to perform the reaction under inert gas at slightly or completely reduced pressure. In the case of use of steam for direct heating of the polymer particles, it is desirable in accordance with the invention to operate the drier under standard pressure or elevated pressure. In this case, it may be advisable to split up the postcrosslinking step into a heating step with steam and a reaction step under inert gas but without steam. This can be achieved in one or more apparatuses. According to the invention, the polymer particles can be heated with steam as early as in the post-crosslinking mixer. The base polymer used may still have a temperature of from 10 to 120° C. from preceding process steps; the postcrosslinker solution may have a temperature of from 0 to 70° C. In particular, the postcrosslinker solution can be heated to reduce the viscosity.

Preferred drying temperatures are in the range of 100 to 250° C., preferably 120 to 220° C., more preferably 130 to 210° C. and most preferably 150 to 200° C. The preferred residence time at this temperature in the reaction mixer or drier is preferably at least 10 minutes, more preferably at least 20 minutes, most preferably at least 30 minutes, and typically at most 60 minutes. Typically, the drying is conducted such that the superabsorbent has a residual moisture content of generally at least 0.1% by weight, preferably at least 0.2% by weight and most preferably at least 0.5% by weight, and generally at most 15% by weight, preferably at most 10% by weight and more preferably at most 8% by weight.

The postcrosslinking may take place under standard atmospheric conditions. “Standard atmospheric conditions” means that no technical precautions are taken in order to reduce the partial pressure of oxidising gases, such as that of atmospheric oxygen, in the apparatus in which the postcrosslinking reaction predominantly takes place (the “postcrosslinking reactor”, typically the drier). However, preference is given to performing the postcrosslinking reaction under reduced partial pressure of oxidising gases. Oxidising gases are substances which, at 23° C., have a vapour pressure of at least 1013 mbar and act as oxidising agents in combustion processes, for example oxygen, nitrogen oxide and nitrogen dioxide, especially oxygen. The partial pressure of oxidising gases is preferably less than 140 mbar, preferably less than 100 mbar, more preferably less than 50 mbar and most preferably less than 10 mbar. When the thermal postcrosslinking is carried out at ambient pressure, i.e. at a total pressure around 1013 mbar, the total partial pressure of the oxidising gases is determined by their proportion by volume. The proportion of the oxidising gases is preferably less than 14% by volume, preferably less than 10% by volume, more preferably less than 5% by volume and most preferably less than 1% by volume.

The postcrosslinking can be carried out under reduced pressure, i.e. at a total pressure of less than 1013 mbar. The total pressure is typically less than 670 mbar, preferably less than 480 mbar, more preferably less than 300 mbar and most preferably less than 200 mbar. When drying and postcrosslinking are carried out under air with an oxygen content of 20.8% by volume, the partial oxygen pressures corresponding to the abovementioned total pressures are 139 mbar (670 mbar), 100 mbar (480 mbar), 62 mbar (300 mbar) and 42 mbar (200 mbar), the respective total pressures being in the brackets. Another means of lowering the partial pressure of oxidising gases is the introduction of non-oxidising gases, especially inert gases, into the apparatus used for postcrosslinking. Suitable inert gases are substances which are present in gaseous form in the postcrosslinking drier at the postcrosslinking temperature and the given pressure and do not have an oxidising action on the constituents of the drying polymer particles under these conditions, for example nitrogen, carbon dioxide, argon, steam, preference being given to nitrogen. The amount of inert gas is generally from 0.0001 to 10 m³, preferably from 0.001 to 5 m³, more preferably from 0.005 to 1 m³ and most preferably from 0.005 to 0.1 m³, based on 1 kg of superabsorbent.

The inert gas, if it does not comprise steam, can be blown into the postcrosslinking drier via nozzles; however, particular preference is given to adding the inert gas to the polymer particle stream via nozzles actually within or just upstream of the mixer, by admixing the superabsorbent with surface postcrosslinker.

It will be appreciated that vapours of cosolvents removed from the drier can be condensed again outside the drier and optionally recycled.

Prior to, during or after the postcrosslinking, in addition to the postcrosslinkers, polyvalent metal ions may be and preferably are added to the surfaces of the inventive superabsorbent or, if no surface postcrosslinking is performed, in their stead. As already stated above, this application of polyvalent metal ions is in principle an (optionally additional) surface postcrosslinking by ionic, noncovalent bonds and is referred to in the context of this invention, for distinction from surface postcrosslinking by means of covalent bonds, as “complexation” with the metal ions in question.

This application of polyvalent metal ions is effected by spray application of solutions of the cations, usually of di-, tri- or tetravalent metal cations. Examples of usable trivalent metal cations are especially the trivalent cations of metals of groups 3 including the lanthanides (especially Sc, Y, La, Ce), 8 (especially Fe), 11 (especially Au) and 13 (especially Al) of the Periodic Table of the Elements. Examples of usable tetravalent cations are especially the tetravalent cations of metals from the lanthanides (especially Ce) and group 4 (especially Ti, Zr, Hf) of the Periodic Table of the Elements. The metal cations can be used either alone or as a mixture with one another. Particular preference is given to the use of trivalent metal cations. Very particular preference is given to the use of aluminium cations.

Polyvalent metal ions are generally added in an amount of at least 0.008% by weight, preferably at least 0.015% by weight and more preferably at least 0.020% by weight, and generally at most 0.15% by weight, preferably at most 0.10% by weight and more preferably at most 0.05% by weight, in each case calculated as the metal and based on the total amount of the anhydrous superabsorbent.

The cations are added in the form of their salts. Among the metal cations mentioned, suitable metal salts are all of those which possess sufficient solubility in the solvent to be used. Particularly suitable metal salts are those with weakly complexing anions, for example chloride, nitrate and sulphate, hydrogensulphate, carbonate, hydrogencarbonate, nitrate, phosphate, hydrogenphosphate, or dihydrogenphosphate. It is also possible to use salts of mono- and dicarboxylic acids, hydroxy acids, keto acids and amino acids, or basic salts. Examples are acetates, propionates, tartrates, maleates and citrates, lactates, malates and succinates or hydroxides. Among the carboxylic salts, 2-hydroxycarboxylic salts such as citrates and lactates are preferred. Examples of particularly preferred metal salts are alkali metal and alkaline earth metal aluminates and hydrates thereof, for instance sodium aluminate and hydrates thereof, aluminium sulphate, aluminium acetate, aluminium propionate, aluminium citrate and aluminium lactate.

The cations and salts mentioned can be used in pure form or as a mixture of different cations or salts. The salts of the di- and/or trivalent metal cation used may comprise further secondary constituents such as still unneutralised carboxylic acid and/or alkali metal salts of the neutralised carboxylic acid. Preferred alkali metal salts are those of sodium and potassium, and those of ammonium. They are typically used in the form of an aqueous solution which is obtained by dissolving the solid salts in water, or is preferably obtained directly as such, which avoids any drying and purification steps. Advantageously, it is also possible to use the hydrates of the salts mentioned, which often dissolve more rapidly in water than the anhydrous salts.

The salt of the polyvalent metal cation can be used in the form of a solution or suspension. Solvents for the metal salts which may be employed are water, alcohols, DMF, DMSO and mixtures of these components. Particular preference is given to water and water/alcohol mixtures, for example water/methanol, water/1,2-propanediol and water/1,3-propanediol.

The treatment of the base polymer with solution of a polyvalent cation is carried out in the same manner as the treatment with surface postcrosslinker, including the drying step. Surface post-crosslinker and polyvalent cation can be sprayed on in a combined solution or as separate solutions. The spray application of the metal salt solution to the superabsorbent particles may either precede or follow the surface postcrosslinking. In a particularly preferred process, the spray application of the metal salt solution is effected in the same step together with the spray application of the crosslinker solution, in which case the two solutions are sprayed on separately in succession or simultaneously via two nozzles, or crosslinker solution and metal salt solution can be sprayed on jointly via one nozzle.

If a drying step is carried out after the surface postcrosslinking and/or treatment with complexing agent, it is advantageous but not absolutely necessary to cool the product after the drying. The cooling can be effected continuously or batchwise; to this end, the product is conveniently conveyed continuously into a cooler arranged downstream of the drier. Any apparatus known for removal of heat from pulverulent solids can be used for this purpose, more particularly any device mentioned above as drying apparatus, except that it is charged not with a heating medium but with a cooling medium, for example with cooling water, such that no heat is introduced into the superabsorbent via the walls nor, according to the construction, via the stirring elements or other heat exchange surfaces, and is instead removed therefrom. Preference is given to the use of coolers in which the product is moved, i.e. cooled mixers, for example shovel coolers, disk coolers or paddle coolers. The superabsorbent can also be cooled in a fluidised bed by injecting a cooled gas such as cold air. The cooling conditions are adjusted so as to obtain a super-absorbent with the temperature desired for further processing. Typically, a mean residence time in the cooler of generally at least 1 minute, preferably at least 3 minutes and more preferably at least 5 minutes, and generally at most 6 hours, preferably at most 2 hours and more preferably at most 1 hour is established, and the cooling performance is such that the product obtained has a temperature of generally at least 0° C., preferably at least 10° C. and more preferably at least 20° C., and generally at most 100° C., preferably at most 80° C. and more preferably at most 60° C.

The surface postcrosslinked superabsorbent is optionally ground and/or sieved in a customary manner. Grinding is typically not required here, but the removal by sieving of agglomerates or fines formed is usually appropriate for establishment of the desired particle size distribution of the product. Agglomerates and fines are either discarded or preferably recycled into the process in a known manner at a suitable point; agglomerates after comminution. The particle sizes desired for surface postcrosslinked superabsorbents are the same as for base polymers. Optionally, the inventive superabsorbents produced by the process according to the invention are provided with further additives. For this purpose, all known additives can be used in the manner known for each in the process according to the invention.

Superabsorbents can be mixed with the compound of formula (I) and any optional additives by any known mixing process. When in solid form, they are incorporated by mixing in substance or as a suspension in a solvent or suspension medium, and, when in dissolved or liquid form, optionally also in solution or liquid form. Due to easier homogeneous distribution, the additives are preferably incorporated into the superabsorbent by mixing as a powder or suspension. This does not necessarily produce a physical mixture separable in a simple manner by mechanical measures. The additives may quite possibly enter into a more definite bond with the super-absorbent, for example in the form of a comparatively firmly adhering surface layer or in the form of particles adhering firmly to the surface of the superabsorbent particles. The mixing of the additives into the known superabsorbent can quite possibly also be understood and referred to as “coating”.

If a solution or suspension is used for coating, the solvent or suspension medium used is a solvent or suspension medium which is chemically compatible both with the superabsorbent and with the additive, i.e. does not enter into any undesired chemical reactions therewith. Typically, water or an organic solvent is used, for example an alcohol or polyol, or mixtures thereof. Examples of suitable solvents or suspension media are water, isopropanol/water, 1,3-propanediol/water and propylene glycol/water, where the mixing ratio by mass is preferably from 20:80 to 40:60. If a suspension medium is used for the stabilisers to be used in accordance with the invention or the inorganic particulate solid, water is preferred. A surfactant can be added to the solution or suspension.

Optional additives are, if they are not added to the monomer mixture or the polymerising gel, generally mixed with the superabsorbent in exactly the same way as the solution or suspension which comprises a surface postcrosslinker and is applied to the superabsorbent for surface postcrosslinking. The additive can be applied as a constituent of the solution applied for surface postcrosslinking or of one of the components thereof to an (as yet) unpostcrosslinked super-absorbent (a “base polymer”), i.e. the additive is added to the solution of the surface postcross-linker or to one of the components thereof. The superabsorbent coated with surface postcross-linker and additives then passes through the further process steps required for surface post-crosslinking, for example a thermally induced reaction of the surface postcrosslinker with the superabsorbent. This process is comparatively simple and economically viable.

When additives are used and sprayed on in the form of dispersions, they are preferably used as aqueous dispersions, and preference is given to additionally applying an antidusting agent to fix the additive on the surface of the superabsorbent. The antidusting agent is then either added directly to the dispersion of the inorganic pulverulent additive; optionally, it can also be added as a separate solution before, during or after the application of the inorganic pulverulent additive by spray application. Most preferred is the simultaneous spray application of postcrosslinking agent, antidusting agent and pulverulent inorganic additive in the postcrosslinking step. In a further preferred process variant, the antidusting agent is, however, added separately in the cooler, for example by spray application from above, below or from the side. Particularly suitable antidusting agents which can also serve to fix pulverulent inorganic additives on the surface of the water-absorbing polymer particles are polyethylene glycols with a molecular weight of 400 to 20 000 g/mol, polyglycerol, 3- to 100-tuply ethoxylated polyols, such as trimethylolpropane, glycerol, sorbitol and neopentyl glycol. Particularly suitable are 7- to 20-tuply ethoxylated glycerol or trimethylolpropane, for example Polyol TP 70® (Perstorp, Sweden). The latter have the advantage, more particularly, that they lower the surface tension of an aqueous extract of the water-absorbing polymer particles only insignificantly.

It is equally possible to adjust the inventive superabsorbent to a desired water content by adding water.

Compounds of formula (I) and all coatings, solids, additives and auxiliaries can each be added in separate process steps, but the most convenient method is usually to add them—if they are not added during the admixing of the base polymer with surface postcrosslinking agent—to the superabsorbent in the cooler, for instance by spray application of a solution or addition in fine solid form or in liquid form.

The inventive superabsorbents generally have a centrifuge retention capacity (CRC, for test method see below) of at least 5 g/g, preferably of at least 10 g/g and more preferably of at least 20 g/g. Typically, it is not more than 40 g/g for surface postcrosslinked superabsorbents, but it is often higher for base polymers.

The inventive superabsorbents have, if they have been surface postcrosslinked, typically an absorption under load (AUL0.7psi, for test method see below) of at least 10 g/g, preferably at least 14 g/g, more preferably at least 18 g/g and most preferably at least 22 g/g, and typically not more than 30 g/g.

The L value of the freshly produced, non-aged superabsorbent (CIE colour number) is typically at least 75, preferably at least 80, more preferably at least 85, and at most 100.

The a value of the freshly produced, non-aged superabsorbent (CIE colour number) is typically from −2.5 to +2.5, preferably from −2.0 to +2.0, more preferably from −1.5 to +1.5.

The b value of the freshly produced, non-aged superabsorbent (CIE colour number) is typically from 0 to 12, preferably from 2 to 11.

According to the relatively high-stress aging tests described below, the inventive superabsorbent, after measurement, has results worsened only to a relatively minor degree for the L and a values compared to the non-aged state, more particularly b values of preferably not more than 13, more preferably not more than 12. A b value above 12 is critical in feminine hygiene articles and ultrathin diapers; a b value of more than 15 is critical even in standard diapers, since this discolouration can be perceived by the consumer on use.

The present invention further provides hygiene articles comprising inventive superabsorbent, preferably ultrathin diapers, comprising an absorbent layer consisting of 50 to 100% by weight, preferably 60 to 100% by weight, more preferably 70 to 100% by weight, especially preferably 80 to 100% by weight and very especially preferably 90 to 100% by weight of inventive super-absorbent, of course not including the envelope of the absorbent layer.

Very particularly advantageously, the inventive superabsorbents are also suitable for production of laminates and composite structures, as described, for example, in US 2003/0181115 and US 2004/0019342. In addition to the hotmelt adhesives described in both documents for production of such novel absorbent structures, and especially the fibers, described in US 2003/0181115, composed of hotmelt adhesives to which the superabsorbent particles are bound, the inventive superabsorbents are also suitable for production of entirely analogous structures using UV-crosslinkable hotmelt adhesives, which are sold, for example, as AC-Resin® (BASF SE, Ludwigshafen, Germany). These UV-crosslinkable hotmelt adhesives have the advantage of already being processable at 120 to 140° C.; they therefore have better compatibility with many thermoplastic substrates. A further significant advantage is that UV-crosslinkable hotmelt adhesives are very safe in toxicological terms and also do not cause any evaporation in the hygiene articles. A very significant advantage in connection with the inventive superabsorbents is the property of the UV-crosslinkable hotmelt adhesives of not tending to yellow during processing and crosslinking. This is especially advantageous when ultrathin or partly transparent hygiene articles are to be produced. The combination of the inventive superabsorbents with UV-crosslinkable hotmelt adhesives is therefore particularly advantageous. Suitable UV-crosslinkable hotmelt adhesives are described, for example, in EP 0 377 199 A2, EP 0 445 641 A1, U.S. Pat. No. 5,026,806, EP 0 655 465 A1 and EP 0 377 191 A2. The inventive superabsorbent can also be used in other fields of industry in which liquids, especially water or aqueous solutions, are absorbed. These fields are, for example, storage, packaging, transport (as constituents of packaging material for water- or moisture-sensitive articles, for instance for flower transport, and also as protection against mechanical effects); animal hygiene (in cat litter); food packaging (transport of fish, fresh meat; absorption of water, blood in fresh fish or meat packaging); medicine (wound plasters, water-absorbing material for burn dressings or for other weeping wounds), cosmetics (carrier material for pharmaceutical chemicals and medicaments, rheumatic plasters, ultrasonic gel, cooling gel, cosmetic thickeners, sunscreen); thickeners for oil/water or water/oil emulsions; textiles (moisture regulation in textiles, shoe insoles, for evaporative cooling, for instance in protective clothing, gloves, headbands); chemical engineering applications (as a catalyst for organic reactions, for immobilisation of large functional molecules such as enzymes, as an adhesive in agglomerations, heat stores, filtration aids, hydrophilic components in polymer laminates, dispersants, liquefiers); as assistants in powder injection moulding, in the building and construction industry (installation, in loam-based renders, as a vibration-inhibiting medium, assistants in tunnel excavations in water-rich ground, cable sheathing); water treatment, waste treatment, water removal (de-icers, reusable sand bags); cleaning; agrochemical industry (irrigation, retention of melt water and dew deposits, composting additive, protection of forests from fungal/insect infestation, retarded release of active ingredients to plants); for firefighting or for fire protection; co-extrusion agents in thermoplastic polymers (for example for hydrophilisation of multilayer films); production of films and thermoplastic mouldings which can absorb water (e.g. films which store rain and dew for agriculture; films comprising superabsorbents for maintaining freshness of fruit and vegetables which are packaged in moist films; superabsorbent-polystyrene co-extrudates, for example for packaging foods such as meat, fish, poultry, fruit and vegetables); or as a carrier substance in active ingredient formulations (pharmaceuticals, crop protection).

The inventive articles for absorption of liquid differ from known examples in that they comprise the inventive superabsorbent.

Also found has been a process for producing articles for absorption of liquid, especially hygiene articles, which comprises using at least one inventive superabsorbent in the production of the article in question. In addition, processes for producing such articles using superabsorbent are known.

Test Methods

The standard test methods described hereinafter that are designated “NWSP” are described in: “Nonwovens Standard Procedures”, 2015 edition, published jointly by EDANA (European Disposables and Nonwovens Association, Avenue Eugene Plasky, 157, 1030 Brussels, Belgium, www.edana.org) and INDA (Association of the Nonwoven Fabrics Industry, 1100 Crescent Green, Suite 115, Cary, N.C. 27518, U.S.A., www.inda.org). This publication is available both from EDANA and from INDA.

All measurements described below should, unless stated otherwise, be conducted at an ambient temperature of 23±2° C. and a relative air humidity of 50±5%. The superabsorbent particles are mixed thoroughly before the measurement unless stated otherwise.

Centrifuge Retention Capacity (CRC)

The centrifuge retention capacity of the superabsorbent is determined according to standard test method NWSP 241.0.R2 (15) “Polyacrylate Superabsorbent Powders—Gravimetric Determination of Fluid Retention Capacity in Saline Solution After Centrifugation”.

Absorbency under a load of 0.7 psi (AUL0.7 psi) (or Absorption under Pressure (AAP))

The absorbency under a load of 4826 Pa (0.7 psi) of the superabsorbent is determined by the standard test method NWSP 242.0.R2 (15) “Polyacrylate Superabsorbent Powders—Gravimetric Determination of Absorption Against Pressure”.

Moisture content of the superabsorbent (residual moisture, water content)

The water content of the superabsorbent particles is determined by standard test method NWSP 230.0.R2 (15) “Polyacrylate Superabsorbent Powders-Estimation of the Moisture Content as Weight Loss Upon Heating”.

Permeability (SFC, “Saline Flow Conductivity”)

The permeability of a swollen gel layer formed by the superabsorbent as a result of liquid absorption is determined under a pressure of 0.3 psi (2069 Pa), as described in EP 0 640 330 A1, as the gel layer permeability of a swollen gel layer of superabsorbent particles, the apparatus described in the aforementioned patent application on page 19 and in FIG. 8 being modified to the effect that the glass frit (40) is not used, and the plunger (39) consists of the same polymer material as the cylinder (37) and now comprises 21 bores of equal size distributed homogeneously over the entire contact area. The procedure and evaluation of the measurement remain unchanged from EP 0 640 330 A1. The flow is detected automatically.

The permeability (SFC) is calculated as follows:

SFC [cm³s/g]=(Fg(t=0)×L0)/(d×A×WP)

where Fg(t=0) is the flow of NaCl solution in g/s, which is obtained using linear regression analysis of the Fg(t) data of the flow determinations by extrapolation to t=0, L0 is the thickness of the gel layer in cm, d is the density of the NaCl solution in g/cm³, A is the area of the gel layer in cm², and WP is the hydrostatic pressure over the gel layer in dyn/cm².

CIE colour number (L, a, b)

The colour analysis is carried out according to the CIELAB method (Hunterlab, volume 8, 1996, book 7, pages 1 to 4) with a “LabScan XE S/N LX17309” colourimeter (HunterLab, Reston, U.S.A.). This method describes the colours via the coordinates L*, a* and b* of a three-dimensional system. L* indicates the brightness, where L*=0 means black and L*=100 white. The values of a* and b* indicate the positions of the colour on the red/green and yellow/blue colour axes respectively, where +a* represents red, −a* represents green, +b* represents yellow and −b* represents blue. The HC60 value is calculated by the formula HC60=L*−3b*. (The asterisks are quite often omitted and only the symbols L, a and b are used.)

This colour space is also described in standard EN ISO 11664-4 “Colorimetry—Part 4: CIE 1976 L*a*b* Colour space”.

Ageing Test

Measurement 1 (initial colour): A plastic dish of internal diameter 9 cm and 1.5 cm height is overfilled with superabsorbent particles which are then smoothed flat with a blade over the edge, and the CIE colour numbers and the HC60 value are determined.

Measurement 2 (after ageing): A plastic dish of internal diameter 9 cm and 1.5 cm height is filled with superabsorbent particles which are then smoothed flat with a blade over the edge. The dish is then placed open into a climate-controlled cabinet heated to 60° C. with constant relative air humidity of 86%. After 21 days have passed, the dish is taken out. After cooling to room temperature, the CIE colour numbers and the HC60 value are determined again.

Accelerated Ageing Test

After coating with the discolouration protection agent solution, superabsorbent particles were immediately placed in 12 mm×55 mm glass petri dishes which allowed for routine observations of Hunter colour data (L, a, b, and H60) and weight monitoring, as well as for pictures to be taken for visual comparison. Initial colour, sample weights, and pictures were recorded. The samples were then placed in an air-tight 5 gallon desiccator containing water as a humectant (SAP: water=1:1 by weight), to ensure that >95% relative humidity was maintained in the chamber throughout the test cycle. The sealed desiccator was placed in a 30 cubic ft. laboratory forced air oven set at 80° C. Periodically, the samples were removed from the desiccator, colour measurements made, and returned to the desiccator for continued treatment. The time passed for the samples to reach L*=75 was determined.

### EXAMPLES

Lignostab® 1198 is a mark of BASF for 1-Oxyl-4-hydroxy-2,2,6,6-tetramethylpiperidine, CAS no.2226-96-2, “4 Hydroxy TEMPO”. The compound is available e.g. from BASF Canada Inc. 100 Milverton Drive, 5th Floor, Mississauga, ON L5R 4H1, Canada, or BTC Europe GmbH, Rheinpromenade 1, 40789 Monheim am Rhein, Germany.

Tinogard® Q is a mark of BASF for 1,4-dihydroxy-2,2,6,6-tetramethylpiperidinium 2-hydroxy-1,2,3-propanetricarboxylate, CAS no 220410-74-2. The compound is available e.g. from BASF SE, Carl-Bosch-Straβe 38, 67056 Ludwigshafen, Germany, or BTC Europe GmbH, Rheinpromenade 1, 40789 Monheim am Rhein, Germany.

HySorb® T9900 and Hysorb® T9400, respectively, are marks of BASF for two of its commercial superabsorbents. The products are available e.g. from BASF SE, Carl-Bosch-Straβe 38, 67056 Ludwigshafen, Germany, or BASF Corporation, 11501 Steele Creek Road, Charlotte, N.C. 28273, U.S.A.

### Example 1

200 g of HySorb® T9900 superabsorbent were set in the plastic bowl of a Bosch MUM 4770 ProfiMixx 47 mixer (Robert Bosch Hausgeräte GmbH, Carl-Wery-Straβe 34, 81739 Munich, Germany) equipped with a metal beater. 0.667 g of a 30 wt. % aqueous solution of Lignostab® 1198 were added (0.1 wt. % of Lignostab® 1198 based on polymer (“bop”, referring to the superabsorbent prior to adding)). The sample was coated with the solution at room temperature by spraying using a syringe during mixing at 180 rpm (speed setting 3). Spraying was followed by further stirring the polymer at speed of 105 rpm for another 5 minutes.

### Example 2

Example 1 was repeated, however, 3.34 g of the 30 wt. % aqueous solution of Lignostab® 1198 were added (0.5 wt. % bop Lignostab® 1198).

### Example 3

Example 1 was repeated, however, 2.0 g of a 10 wt. % aqueous solution of Tinogard Q were added (0.1 wt. % bop Tinogard Q).

### Example 4

Example 3 was repeated, however, 10 g of the 10 wt. % aqueous solution of Tinogard Q were added (0.5 wt. % bop Tinogard Q).

### Example 5

Example 1 was repeated, however, 0.334 g of a 30 wt. % aqueous solution of 4-Hydroxy-TEMPO (Sigma-Aldrich, Germany) were added (0.05 wt. % bop 4-Hydroxy-TEMPO).

### Example 6

Example 5 was repeated, however, 0.667 of the 30 wt. % aqueous solution of 4-Hydroxy-TEMPO were added (0.1 wt. % bop 4-Hydroxy-TEMPO).

The water-absorbing polymer particles coated in examples 1 to 6 and an untreated HySorb® T 9900 sample were subjected to the ageing test. The results are compiled in Table 1.

TABLE 1
Effect of post coating of HySorb ® T9900 on its colour stability in the Ageing Test

| | | Initial colour | | | | After ageing | | | |
|---|---|---|---|---|---|---|---|---|---|
| Example | Coating | L | a | b | H60 | L | a | b | H60 |
| HySorb ®T9900*⁾ | no coating | 94.3 | −0.9 | 5.1 | 78.9 | 48.0 | 11.8 | 18.2 | −6.6 |
| Ex. 1 | 0.1% Lignostab 1198 | 90.7 | 0.0 | 7.8 | 67.4 | 77.7 | 2.1 | 9.8 | 48.2 |
| Ex. 2 | 0.5% Lignostab 1198 | 90.0 | 0.7 | 15.2 | 44.3 | 84.9 | 0.6 | 9.9 | 55.3 |
| Ex. 3 | 0.1% Tinogard Q | 93.8 | −0.6 | 6.2 | 75.2 | 82.3 | 2.6 | 10.2 | 51.7 |
| Ex. 4 | 0.5% Tinogard Q | 93.3 | −0.3 | 7.6 | 70.5 | 82.3 | 2.0 | 11.5 | 47.8 |
| Ex. 5 | 0.05% Hydroxy-TEMPO | 92.6 | −0.1 | 6.9 | 71.9 | 81.9 | 2.8 | 10.2 | 51.2 |
| Ex. 6 | 0.1% Hydroxy-TEMPO | 91.8 | 0.2 | 8.4 | 66.7 | 84.8 | 1.5 | 9.0 | 58.0 |

*⁾comparative

### Example 7

100 g of HySorb® T9400 were coated in a Waring variable speed laboratory blender, Model #38BL54 (Waring Commercial, 314 Ella T. Grasso Ave., Torrington, Conn. 06790, U.S.A.), using a syringe. The coating solution contained no, 0.005 or 0.01 wt. % of Lignostab® 1198 and 40 wt. % of water with respect to dry polymer. The sample was coated with the solution by applying it with a syringe at room temperature over 15 seconds to the polymer while mixing at 2000 rpm. The mixing was continued for an additional 45 seconds after completing the solution addition.

### Example 8

Example 7 was repeated, however, using HySorb® T9900 instead of HySorb® T 9400.

The water-absorbing polymer particles coated in examples 7 and 8 were subjected to the accelerated ageing test. The results are compiled in Table 2.

TABLE 2
Effect of post coating of superabsorbents on their colour
stability in the accelerated ageing test

| Example | Coating | Days to L = 75 |
|---|---|---|
| Ex. 7 a)*⁾ | no coating | 0.8 |
| Ex. 7 b) | 0.005 wt. % Lignostab ® 1198 | 0.8 |
| Ex. 7 c) | 0.01 wt. % Lignostab ® 1198 | 2.0 |
| Ex. 8 a)*⁾ | no coating | 0.8 |
| Ex. 8 b) | 0.005 wt. % Lignostab ® 1198 | 1.1 |
| Ex. 8 c) | 0.01 wt. % Lignostab ® 1198 | 3.2 |

*⁾comparative

The examples demonstrate that the addition of small amounts of TEMPO derivatives improve superabsorbent resistance to colour degradation in hot and humid environments.

1. A superabsorbent comprising at least one compound of the formula (I): 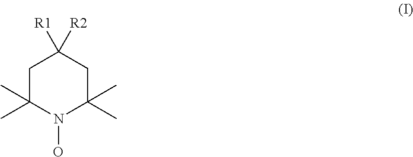
or salt thereof, where R1 and R2 independently are H, organic, or functional substituents.

2. The superabsorbent of claim 1, wherein the compound of formula (I) is selected from the group consisting of (2,2,6,6-Tetramethylpiperidin-1-yl)oxyl, 4-Hydroxi-(2,2,6,6-tetramethylpiperidin-1-yl)oxyl, 4-Acetamido-(2,2,6,6-tetramethylpiperidin-1-yl)oxyl, 4-Acetyl-(2,2,6,6-tetramethylpiperidin-1-yl)oxyl, 4-Oxo-(2,2,6,6-Tetramethylpiperidin-1-yl)oxyL and 1,4-dihydroxy-2,2,6,6-tetramethylpiperidinium 2-hydroxy-1,2,3-propanetricarboxylate.

3. The superabsorbent of claim 1, comprising at least 0.0001 wt. % and at most 5 wt. % of the compound of formula (I), based on the total weight of the dry superabsorbent comprising the compound of formula (I).

4. The superabsorbent of claim 3, comprising at least 0.001 wt. % and at most 1 wt. % of the compound of formula (I), based on the total weight of the dry superabsorbent comprising the compound of formula (I).

5. The superabsorbent of claim 1, wherein the superabsorbent is surface postcrosslinked.

6. The superabsorbent of claim 1, wherein the superabsorbent is surface complexed with polyvalent metal ions.

7. The superabsorbent of claim 6, wherein the superabsorbent is surface complexed with aluminium ions.

8. A process for producing a superabsorbent comprising at least one compound of the formula (I): 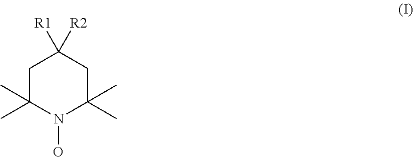
or salt thereof, where R1 and R2 independently are H, organic, or functional substituents, by polymerising an aqueous monomer solution comprising
a) at least one ethylenically unsaturated monomer which bears an acid group and is optionally present at least partly in salt form,
b) at least one crosslinker,
c) at least one initiator,
d) optionally one or more ethylenically unsaturated monomer copolymerisable with the monomer mentioned under a),
e) optionally one or more water-soluble polymer,
the process further comprising:
drying of the resulting polymer,
optionally grinding of the dried polymer and sieving of the ground polymer,
optionally surface postcrosslinking of the dried and optionally ground and sieved polymer, and
mixing the polymer with at least one compound of formula (I).

9. The process according to claim 8, wherein at least one compound of formula (I) is mixed with the polymer after surface postcros slinking.

10. The process according to claim 9, wherein at least one compound of formula (I) is mixed with the polymer in a cooler.

11. An article for absorption of fluids, comprising a superabsorbent defined in claim 1.

12. A process for producing articles for absorption of fluid, wherein the production of the articles comprises addition of a superabsorbent defined in claim 1.
